(19) United States
(12) Patent Application Publication
(10) Pub. No.: US 2005/0033430 A1
(43) Pub. Date: Feb. 10, 2005

(54) Surgical kit and method for providing sterilized equipment for use in spinal surgery

(76) Inventors: Russell Powers, Collierville, TN (US); Bradley Thomas, Memphis, TN (US)

Correspondence Address: Woodard, Emhardt, Moriarty, McNett & Henry LLP;Bank One Center/Tower, Suite 3700, 111 Monument Circle, Indianapolis, IN, 46204-5137, US

(21) Appl. No.: 10/634,206

(22) Filed: Aug. 5, 2003

(51) Int. Cl. A61F 2/44; A61F 2/46; A61B 17/70; A61B 17/88

(52) U.S. Cl. 623/17.11; 606/61; 606/76; 206/370

(57) ABSTRACT

A surgical kit and method for providing sterilized equipment for use in spinal surgery, comprising a spinal implant adapted for engagement with a portion of the spinal column, instrumentation adapted for use in association with the spinal surgery, and packaging adapted to contain the spinal implant and the instrumentation in a sterilized condition prior to the spinal surgery.

### BACKGROUND

Many different types and sizes of implants, devices and instruments are available for treating various diseases, pathologies, injuries or malformations affecting the spine. In the past, the components required for a spinal surgical procedure have been supplied individually to surgical facilities, such as hospitals, trauma or ambulatory centers, medical or research laboratories, and surgical training facilities. Relatively high levels of inventory have been procured and maintained to accommodate the varying requirements associated with a spinal surgical procedure (e.g., anatomical requirements that dictate the selection of a particular size and configuration of implant, device and/or surgical instrument).

As should be appreciated, high inventory levels are expensive to procure and maintain, and are subject to loss, damage and possible theft. Moreover, the cost of even the most basic of surgical instrumentation can be quite high. Additionally, the availability of implants, devices and surgical instrumentation may be scarce, particularly with regard to remote or under-represented surgical facilities. Cleaning, sterilizing and maintaining surgical components can be both time consuming and expensive, particularly with regard to surgical instrumentation that is designed for repeated use. Additionally, cleaning and sterilization procedures may result in significant wait or down time in cases involving back-to-back scheduling of multiple surgical procedures.

Thus, there is a general need in the industry to provide an improved surgical kit and method for delivering sterilized equipment for use in spinal surgery. The present invention meets this need and provides other benefits and advantages in a novel and unobvious manner.

### SUMMARY

The present invention relates generally to a surgical kit and method for delivering sterilized equipment for use in spinal surgery. While the actual nature of the invention covered herein can only be determined with reference to the claims appended hereto, certain forms of the invention that are characteristic of the preferred embodiments disclosed herein are described briefly as follows.

In one aspect of the invention, a surgical kit is provided for use in spinal surgery, comprising a spinal implant adapted for engagement with a portion of the spinal column, instrumentation adapted for use in association with the spinal surgery, and packaging adapted to contain the spinal implant and the instrumentation in a sterilized condition prior to the spinal surgery.

In another aspect of the invention, a method is disclosed for providing sterilized surgical equipment for use in spinal surgery, comprising providing a surgical equipment set including a spinal implant and associated surgical instrumentation, packaging the surgical equipment set within a sealed container, sterilizing the surgical equipment set, and delivering the surgical equipment set to a surgical site.

Further aspects of the invention will become apparent from the drawings and description that follow.

### BRIEF DESCRIPTION OF THE DRAWINGS

FIG. 1 is a perspective view of a surgical kit for use in spinal surgery according to one embodiment of the invention, as shown with the kit packaging seals removed for clarity.

FIG. 2 is an exploded view of the packaging associated with the surgical kit illustrated in FIG. 1.

FIG. 3 is a surgical template according to one embodiment of the invention for use in association with the surgical kit illustrated in FIG. 1.

FIG. 4 is an exploded view of a surgical instrument according to one embodiment of the invention for use in association with the surgical kit illustrated in FIG. 1.

FIG. 5 is a spinal fixation plate according to one embodiment of the invention for use in association with the surgical kit illustrated in FIG. 1, as anchored to an anterior cervical region of the spinal column.

FIG. 6 is a lateral side view of the embodiment of the invention illustrated in FIG. 5.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, such alterations and further modifications in the illustrated devices, and such further applications of the principles of the invention as illustrated herein being contemplated as would normally occur to one skilled in the art to which the invention relates.

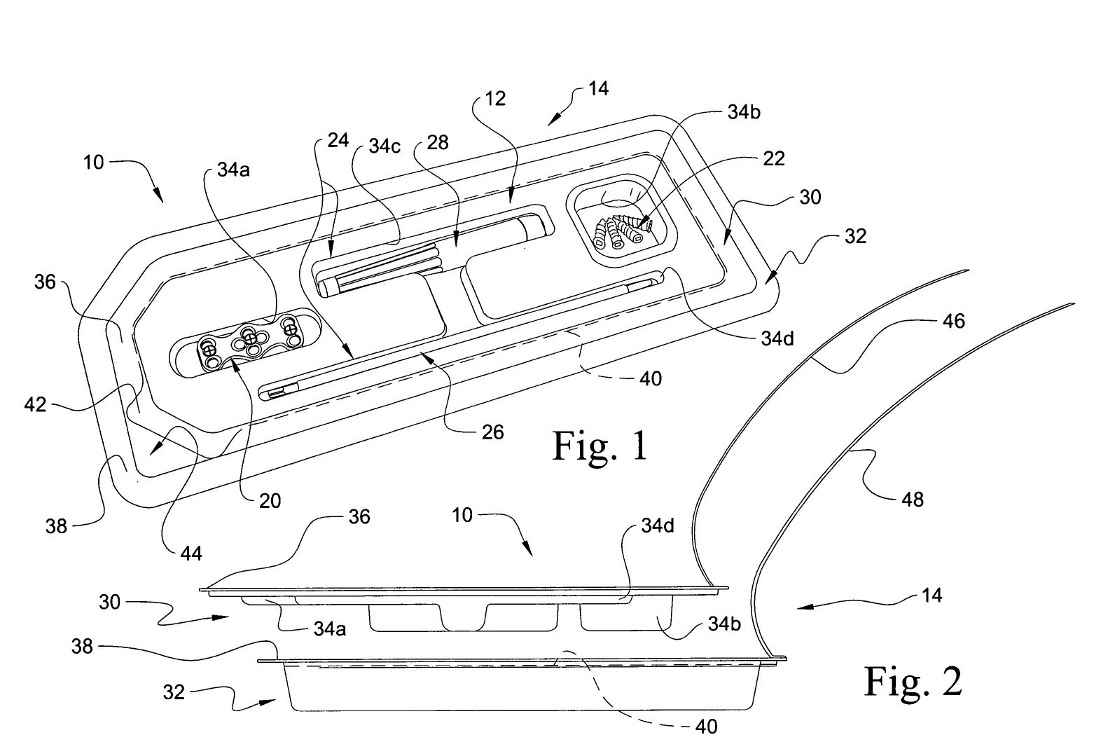

Referring to FIG. 1, shown therein is a surgical kit 10 for use in association with spinal surgery according to one embodiment of the invention. The surgical kit 10 is generally comprised of a surgical equipment set 12 and packaging 14 for containing and maintaining the surgical equipment set 12 in a sterilized condition prior to surgery. The surgical equipment set 12 may include various sizes and configurations of spinal implants and associated components and/or instruments suitable for use in a spinal surgical procedure. In the illustrated embodiment, the surgical equipment set 12 includes a spinal implant 20, a number of bone anchors 22 for securing the spinal implant 20 to a portion of the spinal column, and surgical instrumentation 24 for use in securing the spinal implant 20 to the spinal column. However, as will be discussed below, the surgical equipment set 12 may include other types and configurations of spinal implants and associated components and/or instruments suitable for use in a spinal surgical procedure.

In a further embodiment of the invention, the surgical equipment set 12 includes all of the implants, components and/or instruments to perform a designated spinal surgical procedure. In this manner, the surgical kit 10 is self-contained to include all of the specialized equipment required to perform a designated spinal surgical procedure. It should be understood, however, that general surgical equipment is also contemplated for inclusion in the surgical kit 10, such as, for example, scalpels, retractors, local anesthesia and associated devices, and/or other types of general surgical components and instrumentation.

As will be discussed in further detail below, in one embodiment of the invention, the spinal implant 20 is configured as an elongate member for use in immobilizing and/or stabilizing a portion of the spinal column. In the illustrated embodiment of the invention, the spinal implant 20 is configured as a spinal plate. However, it should be understood that the spinal implant 20 can take on other configurations, such as, for example, a rod, a cable, or any type of elongate element suitable for use in immobilizing and/or stabilizing a portion of the spinal column. It should also be understood that other types of spinal implants are also contemplated for use in association with the present invention, such as, for example, interbody implants or any other type of implant suitable for use in association with spinal surgery. It should further be understood that a bone-growth promoting substance or material may be included in the surgical kit 10 to promote fusion with the spinal implant and/or between the adjacent vertebrae. Such bone-growth substances/materials may include but are not limited to natural bone material including bone chips or bone marrow, a demineralized bone matrix (DBM), mesenchymal stem cells, a bone morphogenic protein (BMP), a LIM mineralization protein (LMP), or any other suitable bone-growth promoting substance or material.

As will also be discussed in further detail below, in the illustrated embodiment of the invention, the bone anchors 22 are configured as bone screws suitable for use in engaging the spinal plate 20 to a portion of the spinal column. In one embodiment, the bone screws 22 are configured as self-cutting screws, thereby eliminating the requirement for cutting instruments, such as, for example, drilling, tapping and/or guiding instruments to anchor the bone screws 22 within vertebral bone. However, it should be understood that other types and configurations of bone screws are also contemplated for use in association with the present invention. It should also be understood that the bone anchors 22 can take on other configurations, such as, for example, bolts, hooks, staples or other types of devices suitable for securing the spinal implant 20 to a portion of the spinal column.

As will additionally be discussed in further detail below, in the illustrated embodiment of the invention, the surgical instrumentation 24 comprises a driver instrument configured to engage and drive the bone screws 22 into vertebral bone and/or for engaging, driving or manipulating other components or elements associated with the spinal plate 20. However, it should be understood that other types and configurations of surgical instruments are also contemplated for use in association with the present invention, including, for example, distractors, insertion instruments, holders, guides, cutting instruments such as saws, drills, taps, trephines, curettes and chisels, and/or other types and configurations of instruments suitable for use in association with spinal surgery.

Referring collectively to FIGS. 1 and 2, in one embodiment of the invention, the packaging 14 generally comprises an inner packaging container 30 and outer packaging container 32. The inner packaging container 30 serves to contain and * maintain the sterility of the surgical equipment set 12 until such time as the equipment set 12 is to be accessed, which will typically occur just prior to surgery or during surgery. Similarly, the outer packaging container 32 serves to contain and maintain the sterility of the inner packaging container 30 until such time as the surgical equipment set 12 is to be accessed. However, it should be understood that in other embodiments of the invention, the packaging 14 need not necessarily include the outer packaging container 32. Additionally, although not specifically illustrated in the Figures, the surgical kit 10 may be placed in an outer enclosure, such as, for example, a box, carton, envelope, bag or other suitable types of containers or enclosures, to aid in the delivery, storage and/or identification of the surgical kit 10.

In one embodiment of the invention, the inner and outer packaging containers 30, 32 are generally configured as trays, with the inner tray 30 defining a number of compartments or blisters 34a, 34b, 34c and 34d sized and configured to receive various components of the surgical equipment set 12 therein, and with the outer tray 32 sized and configured to receive the inner tray 30 therein. In one embodiment, the inner and outer packaging trays 30, 32 are formed of a material that provides for direct visualization of the surgical equipment set 12 contained therein, such as, for example, a clear, transparent or translucent material. In this manner, the contents of the surgical equipment set 12 can be confirmed or verified without having to open the packaging 14 and without compromising the sterility of the surgical kit 10. In a specific embodiment, the inner and outer packaging trays 30, 32 are formed of a substantially clear, plastic material. However, other types of packaging materials suitable for maintaining the sterility of the surgical kit 10 are also contemplated for use in association with the present invention.

In a further embodiment of the invention, the inner and outer packaging trays 30, 32 include peripheral outer lips 36 and 38, respectively, that provide for convenient handling of the trays 30, 32 and for packaging considerations. As illustrated in FIG. 1, the peripheral lip 36 of the inner packaging tray 30 rests on a shoulder 40 defined about the inner periphery of the outer packaging tray 32 to securely nest the inner tray 30 within the outer tray 32. The peripheral lip 36 of the inner tray 30 also defines a recessed or inset portion 42 that is offset from the side wall of the outer tray 32 so as to define an open area 44. As should be appreciated, the open area 44 allows for convenient grasping of the peripheral lip 36 of the inner tray 30 to facilitate removal of the inner tray 30 from the outer tray 32.

In a further embodiment of the invention, as illustrated in FIG. 2, the inner tray 30 includes a seal or cover 46 that is removably attached to the peripheral lip 36 to maintain the sterility of the surgical equipment set 12 contained therein. The seal 46 may be removed or pealed away from the inner tray 30 to provide selective access to the surgical equipment set 12. Similarly, the outer tray 32 includes a seal or cover 48 that is removably attached to the peripheral lip 38 to maintain the sterility of the inner tray 30. The seal 48 may be removed or pealed away from the outer tray 32 to provide selective access to the inner tray 30.

In one embodiment of the invention, the surgical kit 10 is sterilized as a complete unit following sealing of the surgical equipment set 12 within the inner packaging container 30 and sealing of the inner packaging container 30 within the outer packaging container 32. Sterilization of the surgical kit 10 may be accomplished via a radiating technique, such as, for example, by exposing the surgical kit 10 to gamma radiation. However, it should be understood that the surgical kit 10 may be sterilized by outer suitable sterilization techniques. It should also be understood that the surgical equipment set 12 may be sterilized prior to being sealed within the inner packaging container 30, and that the inner packaging container 30 may be sterilized prior being sealed within the outer tray 30.

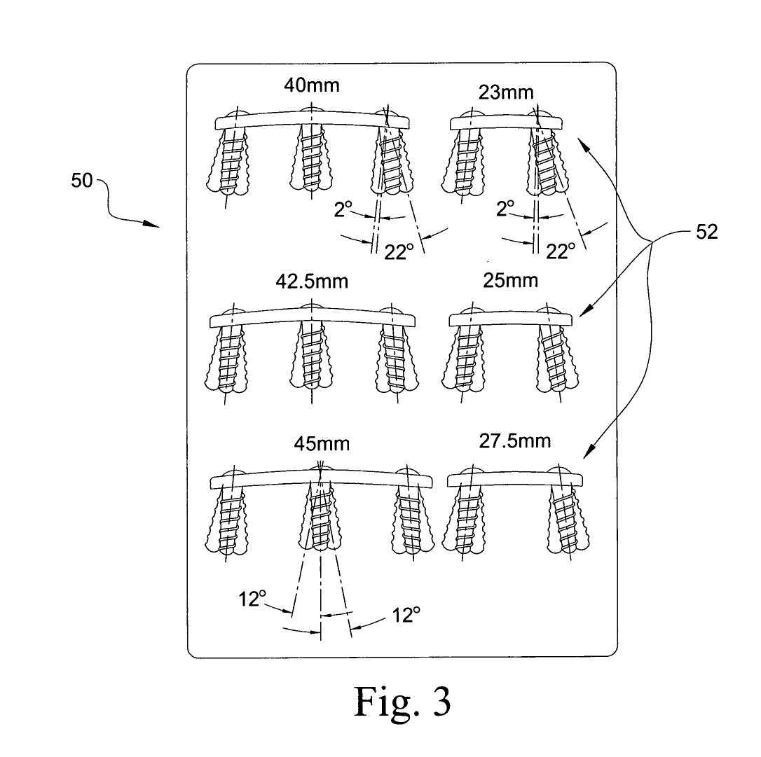

Referring now to FIG. 3, shown therein is a template 50 according to one embodiment of the invention for use in association with the surgical kit 10. The template 50 includes a number of graphical images or illustrations 52 that correspond to various sizes and configurations of spinal implants and devices to be used in association with a designated spinal surgical procedure. In one embodiment, the template 50 illustrates lateral views of various sizes and configurations of spinal plates and bone anchors that correspond to select sizes and configurations of the spinal plate 20 and bone anchors 22 included in a particular surgical kit 10. Specifically, the template 50 includes a number of images 52 of spinal plates having varying lengths and plate configurations. Additionally, the images 52 illustrate bone anchors having a certain length and which are adapted to pivot relative to the spinal plate within a range of angular orientations. In other embodiments, the bone anchors and/or the spinal plate may be adapted to allow for relative translational movement therebetween, or a combination of relative pivotal and translational movement therebetween. It should also be understood that the bone anchors may be adapted for placement in a predetermined fixed position and/or orientation relative to the spinal plate.

In the illustrated embodiment of the invention, the surgical kit 10 includes a spinal plate 20 having a size selected from a range of plate sizes; namely, 23 mm, 25 mm, 27.5 mm, 40 mm, 42.5 mm and 45 mm. These particular plate sizes were selected to satisfy a broad range of spinal surgical procedures and applications. For example, in the illustrated embodiment, the designated plate sizes were selected to satisfy the requirements for approximately 80% of the spinal surgical procedures and applications that utilize the particular configuration of the spinal plate 20. Additionally, in the illustrated embodiment of the invention, the surgical kit 10 is equipped with variable-angle bone screws 22 have a length of about 13 mm. This particular size and configuration of bone screw was selected for use in association with the designated sizes and configurations of the spinal plate 20, and to satisfy the requirements for a broad range of spinal surgical procedures and applications. Although specific sizes and configurations of the spinal plate 20 and bone anchors 22 have been illustrated and described herein, it should be understood that the designated sizes and configurations of the spinal plates and bone anchors are exemplary, and do not in any way limit the scope of the present invention. It should also be understood that the components included in the surgical kits 10 are selected to maximize usage of the spinal kits 10 in association with a broad range of spinal surgical procedures and applications, and to minimize the overall inventory levels of the surgical equipment required to perform such surgical procedures and applications.

As should be appreciated, each of the images 52 illustrated on the template 50 corresponds to a specific size and configuration of the spinal implant 20 and associated components, such as the bone anchors 22, included in a particular surgical kit 10. Accordingly, the template 50 may be used to aid the surgeon or other medical personnel in the selection of a surgical kit 10 which includes the appropriate size and configuration of spinal plate 20 and bone screws 22 required to satisfy the particular requirements of the spinal surgical procedure being performed. Specifically, the template images 52 may be compared to a representation of the portion of the spinal column being treated to determine whether the size and configuration of the spinal plate 20 and/or the bone anchors 22 included in the surgical kit 10 will satisfy the particular requirements of the designated spinal surgical procedure.

In one embodiment of the invention, the representation against which the template images 52 are compared is an x-ray image. However, it should be understood that other suitable devices and techniques may be used to provide a visual representation against which the template images 52 are compared, such as, for example, magnetic resonance imaging (MRI), ultrasound imaging, or other types of imaging techniques. As should be appreciated, x-ray representations are sometimes magnified or reduced by a factor greater than or less than 100%. Accordingly, the template 50 is preferably marked to indicate the magnification factor attributable to the template images 52 (e.g., 95%, 100%, 115%, etc.). As a result, the surgeon or other medical personnel can match the magnification factor associated with the template 50 with the magnification factor associated with the x-ray representation to ensure proper selection of the appropriate size and/or configuration of the spinal implant 20 and/or bone anchors 22. It should be understood that the surgical kit 10 may be provided with a single template 50 to accommodate x-ray representations having a select magnification factor, or with multiple templates 50 to accommodate x-ray representations having a range of magnification factors. In another embodiment of the invention, a conversion table may be included with the surgical kit 10 to aid in calculating the appropriate conversion factor associated with the magnification factor attributable to the template 50 and/or the x-ray representation.

In a further embodiment of the invention, the template 50 is attached to the exterior of the outer packaging container 32 or to the exterior of the outer box or enclosure (not shown) within which the surgical kit 10 is contained. As a result, the template 50 can be accessed by a surgeon or other medical personnel without having to open the packing 14. Accordingly, the sterility of the inner packaging container 30 and/or the surgical equipment 12 contained therein is not compromised in the event that the particular size and/or configuration of the spinal implant 20 and/or bone anchors 22 included with the surgical kit 10 fail to satisfy the particular requirements of the designated spinal surgical procedure. In one embodiment, the template 50 is contained within a plastic bag or envelope (not shown), which is in turn attached to the exterior of the outer packaging container 32 or to the exterior of the outer box or enclosure. However, it should be understood that other suitable devices and techniques may be used for attaching or otherwise including the template 50 with the surgical kit 10.

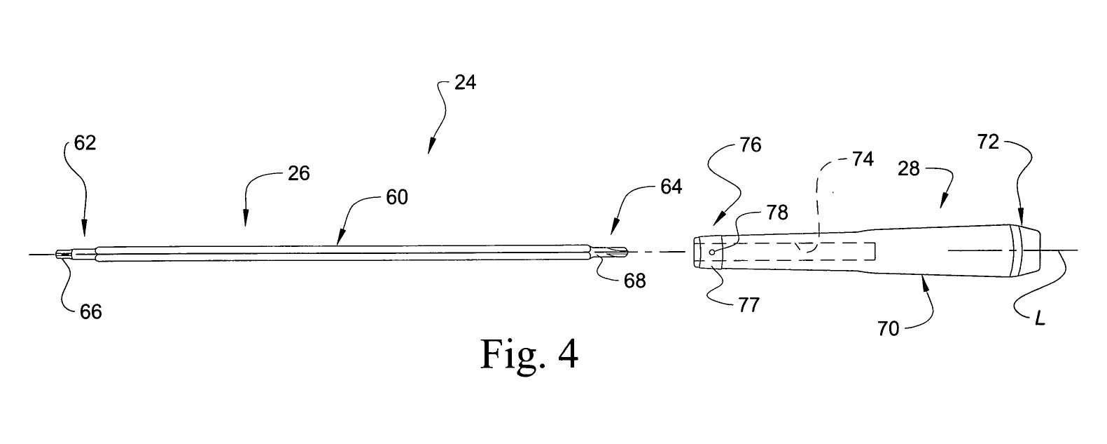

Referring to FIG. 4, shown therein is an exploded view of the driver instrument 24. The driver instrument 24 extends generally along a longitudinal axis L and includes a shaft portion 26 and a handle portion 28. In one embodiment of the invention, the shaft 26 and the handle 28 are configured as separate pieces that are assembled or otherwise integrated to form the driver instrument 24. Since the shaft 26 and handle 28 are configured as separate pieces, the overall size (e.g., the length) of the packaging 14 may be reduced. Additionally, as will be discussed in further detail below, the two-piece configuration of the driver instrument 24 allows the end portions of the shaft 26 to be reversed relative to the handle 28 to provide the driver instrument 24 with alternative end configurations. However, it should be understood that in other embodiments of the invention, the shaft 26 and handle 28 may be formed integral with one another to form single-piece driver instrument 24.

In one embodiment of the invention, the shaft 26 includes a central portion 60 and opposite first and second end portions 62 and 64. In a specific embodiment, the central portion 60 has a hexagonal configuration; however, other suitable shapes and configurations are also contemplated. In another embodiment, the first and second end portions 62 and 64 have shaped configurations suitable for engagement with various elements and components associated with the spinal plate 20 and/or the bone anchors 22, the details of which will be discussed below. In this manner, the driver instrument 24 is multi-functional in that the shaft 26 is capable of engaging, driving, displacing and/or manipulating one or more elements or components associated with the spinal plate 20 and/or the bone anchors 22.

In the illustrated embodiment of the driver instrument 24, the first end portion 62 of the shaft 26 defines a driving tip 66 sized and configured for insertion within a tool receiving recess defined by the bone screws 22. In a specific embodiment, the driving tip 66 has a hexagonal configuration. However, it should be understood that other suitable shapes and configuration of the driving tip 66 are also contemplated for use in association with the driver instrument 24. The second end portion 64 of the shaft 26 defines a driving tip 68 sized and configured for insertion within a tool receiving recess defined by another element or component associated with the spinal plate 20, the details of which will be discussed below. In a specific embodiment, the driving tip 68 has a cross-shaped or Phillips-type configuration. However, it should be understood that other suitable shapes and configuration of the driving tip 68 are also contemplated for use in association with the driver instrument 24.

In one embodiment of the invention, the handle 28 of the driver instrument 24 includes a main body portion 70 and a distal end portion 72, with the distal end portion 72 being rotatably coupled to the main body portion 70 to allow for relative rotation therebetween about the longitudinal axis L. This particular configuration of the handle 28 facilitates ease of use with regard to single-handed driving rotation of the driver instrument 24. More specifically, the distal end portion 72 may be grasped in the user's palm or between the user's little finger and palm, while the main body portion 70 is rotated between the user's thumb and index finger to correspondingly rotate the shaft 26. However, it should be understood that other configurations of the handle 28 are also contemplated for use in association with the driver instrument 24, including single-piece handle configurations.

The main body portion 70 of the handle 28 defines an axial opening 74 that is sized and configured to receive a portion of the shaft 26 therein. In one embodiment, the axial opening 74 has a shape corresponding to an outer profile of the shaft 26. In a specific embodiment, the axial opening 74 has a hexagonal shape sized to receive the hexagonally-shaped central portion 60 of the shaft 26 therein. In this manner, the shaft 26 is engaged with the handle 28 to substantially prevent relative rotation therebetween. However, it should be understood that other suitable shapes and configurations of the axial opening 74 and the central shaft portion 60 are also contemplated, such as, for example, rectangular, polygonal, circular or elliptical configurations. It should also be understood that other means for preventing relative rotation between the shaft 26 and the handle 28 are also contemplated, such as, for example, via pinning or fastening engagement.

In another embodiment of the driver instrument 24, the handle 28 includes a retaining mechanism 76 configured to selectively maintain axial engagement between the shaft 26 and the handle 28. In a specific embodiment, the retaining mechanism 76 includes a ring or band 76 disposed about the handle 28, and a detent device 78 supported by the ring 76 and in communication with the axial opening 74. In another specific embodiment, the detent device 78 comprises a loaded ball bearing that operates to allow the central shaft portion 60 to be slidably inserted into and selectively removed from the axial opening 74. The detent device 78 also functions to exert a transverse force against the central shaft portion 60 to aid in selectively maintaining the shaft 26 in axial engagement with the handle 28. However, it should be understood that other means for maintaining axial engagement between the shaft 26 and the handle 28 are also contemplated, such as, for example, via pinning, fastening or threading engagement.

In a further embodiment of the invention, the instrumentation 24 included with the surgical kit 10 is designed for planned disposable upon use in association with a limited number of spinal surgeries. In a specific embodiment, the instrumentation 24 is designed for a single use in association with a single spinal surgery. However, it should be understood that in other embodiments, the instrumentation 24 may be designed for multiple spinal surgeries, including two, three, of four or more spinal surgeries. In other words, the instrumentation 24 may be designed to have a predetermined life span for use in association with a limited number of spinal surgeries, after which the instrumentation 24 is subjected to disposal. In instances where the instrumentation 24 included with the surgical kit 10 is designed for a single use, immediate disposal eliminates the requirements and costs associated with cleaning, sterilizing, repackaging, and/or storing the instrumentation for repeat use.

In one embodiment of the invention, at least a portion of the instrumentation is subject to degradation upon exposure to a sterilization procedure, such as, for example, autoclaving or other sterilization techniques. As will be discussed below, in one embodiment, at least a portion of the instrumentation 24 is subject to deformation upon exposure to a sterilization procedure. In another embodiment, at least a portion of the instrumentation 24 is subject to discoloration upon exposure to a sterilization procedure. However, other types of degradation are also contemplated as falling within the scope of the invention, such as, for example, bending, weakening, cracking, breaking, pitting, flaking, disintegrating, dissolving, or any other form of degradation. As will also be discussed below, degradation of at least a portion of the instrumentation 24 may occur gradually upon exposure to multiple sterilization procedures, or immediately upon exposure to a single sterilization procedure.

In one embodiment of the invention, the shaft 26 of the driver instrument 24 is formed of a metallic material, such as, for example, steel or another material suitable for surgical applications, and is coated with a material that has a propensity to discolor or blemish if the shaft 26 is subjected to a sterilization procedure, such as, for example, autoclaving. In a specific embodiment, the shaft 26 is coated with a hard chrome material that tends to discolor or blemish and form spots or splotches upon exposure to a sterilization procedure. However, other suitable materials or material coatings are also contemplated for use in association with the shaft 26. It should be appreciated that discoloration or blemishing of the shaft 26 tends to discourage or deter further use of the shaft 26, and accordingly tends to limit use of the instrumentation 24 to a single surgical procedure.

In a further embodiment of the invention, the handle 28 is formed of a plastic or polymeric material, such as, for example, a polycarbonate material, which has a propensity to deform if the handle 28 is subjected to a sterilization procedure, such as, for example, autoclaving. In one embodiment, the axial opening 74 of the handle 28 and/or the retaining mechanism 76 deforms, degrades or is otherwise altered upon exposure to a sterilization procedure so as to prevent the shaft 26 from being inserted into or removed from the axial opening 74. Deformation of the handle 28 tends to discourage or deter further use, and accordingly tends to limit use of the instrumentation 24 to a limited number of surgical procedures.

In a specific embodiment of the invention, the shaft 26 is designed to discolor or blemish and the handle 28 is designed to deform after being subjected to a single sterilization attempt, thereby tending to limit use of the instrumentation 24 to a single surgical procedure. However, in other embodiments, the shaft 26 may be designed to gradually discolor or blemish, and/or the handle 28 may be designed to gradually deform upon exposure to multiple sterilization procedures, thereby allowing for a controlled number of multiple uses of the instrumentation 24.

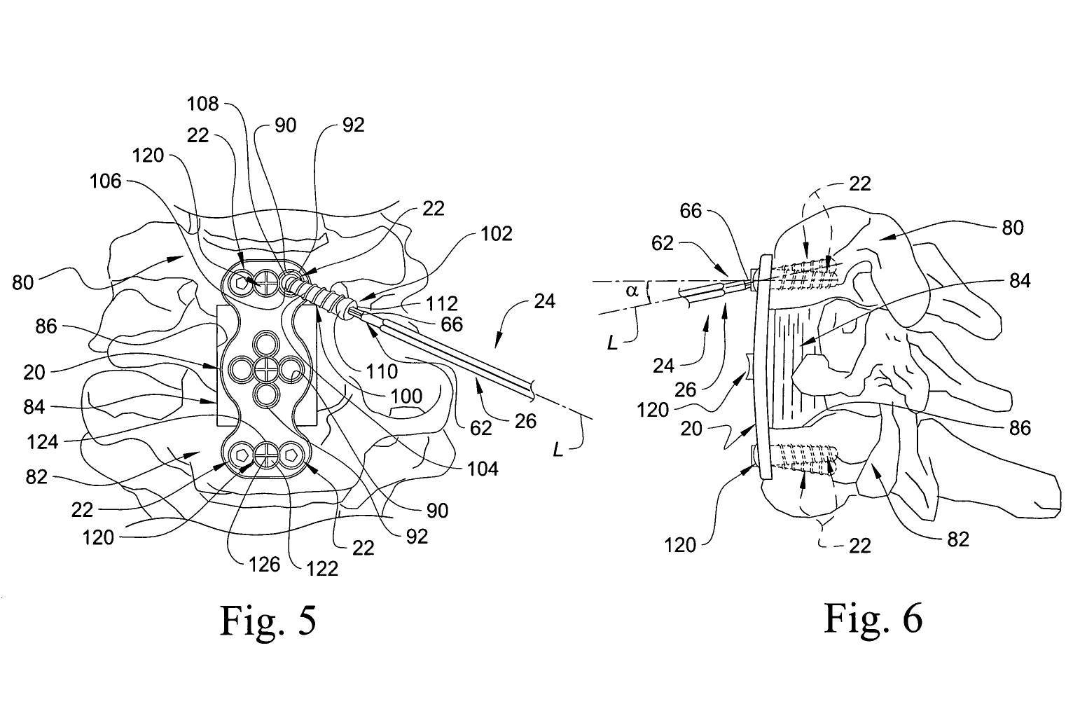

Referring to FIGS. 5 and 6, shown therein is the spinal plate 20 anchored to the cervical region of the spinal column via the bone screws 22, and more specifically to an anterior aspect of upper and lower cervical vertebrae 80 and 82. However, it should be understood that the surgical kit 10 may be used in association with other regions of the spine, such as, for example, the thoracic, lumbar, lumbo sacral, sacral and/or occipital regions of the spine. It should also be understood that the spinal plate 20 may be applied to other aspects of the vertebrae and via other surgical approaches, such as, for example, antero-lateral, oblique and posterior surgical approaches. In the illustrated embodiment of the invention, the spinal plate 20 is sized and configured to span across three vertebral levels. However, it should be understood that the spinal plate 20 may be sized and configured to extend across any number of vertebral levels, including a single vertebral level, two vertebral levels or four or more vertebral levels.

In the illustrated embodiment of the invention, an interbody implant 84 is positioned within an intervertebral space or opening 86 extending between the upper and lower cervical vertebrae 80, 82 to provide stabilization and/or support to the portion of the spinal column being treated. However, it should be understood that an interbody implant 84 need not necessarily be used in association with the spinal plate 20 to provide stabilization and support to the spinal column. In the illustrated embodiment, the interbody implant 84 is configured as a bone graft. However, other types and configurations of interbody implants and associated devices are also contemplated for use in association with the spinal plate 20, such as, for example, bone dowels, struts, spacers, push-in type cages, screw-in type cages, tapered cages, mesh cages, cages filled with bone graft and/or graft substitute material, articulating implants, or other types of suitable interbody implants. It should be understood that the spinal plate 20 and/or the interbody implant 84 may be used in association with fusion-type applications that promote interbody fusion between adjacent vertebrae, in association with spacer-type applications that generally serve to maintain a spacing between adjacent vertebrae without fusion, and/or in association with articulating-type applications that serve to substantially restore normal biomechanical motion to the portion of the spinal column being treated.

Although the illustrated embodiment of the surgical kit 10 does not include the interbody implant 84, it should be understood that in other embodiments of the invention, the interbody implant 84 may be included with the surgical kit 10. It should also be understood that the interbody implant 84 may be incorporated into a separate surgical kit which includes devices and/or instruments for use in association with forming the intervertebral space 86, preparing the interbody implant 84 for implantation, and/or inserting the interbody implant 84 into the intervertebral space 86. Such interbody implant kits or the surgical kit 10 may also include an amount of a bone-growth promoting substance or material to promote fusion with the interbody implant and/or between the adjacent vertebrae. Such bone-growth substances/materials may include but are not limited to natural bone material including bone chips or bone marrow, a demineralized bone matrix (DBM), mesenchymal stem cells, a bone morphogenic protein (BMP), a LIM mineralization protein (LMP), or any other suitable bone-growth promoting substance or material.

As illustrated in FIG. 5, the spinal plate 20 includes a number of bone anchor openings 90 that are sized and configured to receive respective ones of the bone anchors 22. Each of the bone anchor openings 90 includes a spherical-shaped recessed portion 92, the purpose of which will be discussed below. In the illustrated embodiment, the spinal plate 20 includes a pair of bone anchor openings 90 located adjacent opposite ends of the spinal plate 20 to provide for secure anchoring of the spinal plate 20 to the upper and lower vertebrae 80, 82. A number of bone anchor openings 90 may also be located along the mid-portion of the spinal plate 20 to provide for optional anchoring of the spinal plate 20 to the interbody implant 84 and/or to the intermediate vertebra disposed between the upper and lower vertebrae 80, 82. The bone anchor openings 90 located along the mid-portion of the spinal plate 20 may also serve to provide for direct visualization of a portion of the interbody implant 84 and/or the intermediate vertebra. Additional details regarding the spinal plate 20 and other devices and components associated therewith are illustrated and described in U.S. Pat. No. 6,152,927 to Farris et al., the contents of which are hereby incorporated by reference.

The spinal plate 20 is secured to the upper and lower vertebrae 80, 82 via a plurality of the bone screws 22. In the illustrated embodiment of the invention, four bone screws 22 are used to engage the spinal plate 20 to the upper and lower vertebrae 80, 82. However, it should be understood that any number of bone screws 22 may be used, including two, three, or five or more bone screws 22. The bone screw 22 generally include a threaded shank portion 100 sized to pass through a respective bone anchor opening 90 in the spinal plate 20, and a head portion 102 configured to abut the spinal plate 20. Although a specific type and configuration of bone screw has been illustrated and described herein, it should be understood that other types and configurations of bone screws are also contemplate for use in association with the present invention.

In one embodiment of the invention, the threaded shank portion 100 of the bone screw 22 defines external threads 104 adapted to engage bone, and more specifically cortical and/or cancellous vertebral bone. As discussed above, the bone screws 22 may be configured as self-cutting screws, thereby eliminating the requirement for additional instrumentation, such as, for example, drilling, tapping and/or guiding instruments to secure the spinal plate 20 to the vertebrae 80, 82. In a specific embodiment, the threaded shank 100 includes a fluted or recessed area 106 extending across a number of the threads 104 to facilitate self-drilling and/or self-tapping into bone. In a further embodiment, the distal end portion 108 of the threaded shank 100 may be tapered and/or pointed to facilitate penetration into bone.

In another embodiment of the invention, the head portion 102 of the bone screws 22 includes a spherical-shaped surface 110 that is substantially complementary to the spherical-shaped recessed portion 92 defined by the bone anchor openings 90. Engagement of the spherical-shaped surface 110 of the screw head 102 with the spherical-shaped recessed portion 92 of the openings 90 allows the bone screws 22 to be pivotally positioned relative to the spinal plate 20 within a range of angular orientations a (FIG. 6). This variable-angle capability allows the surgeon to engage the bone screws 22 to the upper and lower vertebrae 80, 82 within a range of angular orientations, thereby providing greater flexibility in securing the spinal plate 20 to the particular portion of the spinal column being treated. Moreover, this variable-angle capability permits a limited degree of micro-motion or translation between bone screws 22 and the spinal plate 20 which may prevent or at least reduce the build-up of load stresses.

In a further embodiment of the invention, the head portion 102 of the bone screws 22 defines a tool receiving recess 112 (FIG. 5) configured to receive an end portion of the driver instrument 24 therein to facilitate driving of the bone screws 22 into bone. In one specific embodiment, the tool receiving recess 112 has a hexagonal configuration sized and configured to engagingly receive the hexagonally-shaped tip 66 of the driver instrument 24 therein. However, it should be understood that other suitable shapes and configuration of the tool receiving recess 112 are also contemplated for use in association with the bone anchor 22.

As illustrated in FIG. 5, the spinal plate 20 includes a number of retaining devices 120 configured for engagement with the bone screws 22 to prevent the bone screws 22 from loosening and backing out of vertebral bone. In one embodiment, the retaining device 120 is configured as a threaded fastener 122 and a washer 124. The fastener 122 includes a threaded shank configured for threading engagement within a threaded aperture (not shown) defined by the spinal plate 20, and a head portion configured to abut against the washer 124 to engage the washer 124 against the head portions 102 of the bone screws 22. The head portion of the fastener 122 defines a tool receiving recess 126 configured to receive an end portion of the driver instrument 24 therein to facilitate driving of the fastener 122 into the threaded aperture (not shown) in the spinal plate 20. In one embodiment, the tool receiving recess 126 has a cross-shaped or Phillips-type configuration that is sized and configured to engagingly receive the cross-shaped tip 68 of the driver instrument 24 therein. However, it should be understood that other suitable shapes and configuration of the tool receiving recess 126 are also contemplated for use in association with the fastener 122.

Having described the basic features and components of the spinal plate 20 and the bone anchors 22, reference will now be made to a technique for engaging the spinal plate 20 to the spinal column according to one embodiment of the invention. Initially, the portion of the patient's spinal column being treated is x-rayed to provide a visual representation of the spinal anatomy. The template 50 is then overlaid with the x-ray representation of the patient's spinal column to determine the appropriate size and/or configuration of the spinal plate 20 and/or bone anchors 22 to be used in association with the designated spinal surgical procedure. Since the template 50 is attached to the exterior of the outer packaging container 32 or to the exterior of an outer box or enclosure (not shown) within which the surgical kit 10 is contained, the template 50 can be accessed by a surgeon or other medical personnel without opening the packing 14. As a result, the sterility of the inner packaging container 30 and the surgical equipment set 12 is maintained.

Upon verification of the correct size and/or configuration of the spinal plate 20 and/or bone anchors 22 to be used in association with the designated surgical procedure, the seal 48 on the outer packaging container 32 (FIG. 2) may be removed to provide access to the inner packaging container 30. Since the inner container 30 is maintained in a sterilized condition, it may be introduced directly into a sterile operating room environment. Following removal of the inner container 30 from the outer container 32, the seal 46 on the inner container 30 may be removed to provide access to the surgical equipment set 12, and more specifically to the spinal plate 20, the bone anchors 22 and the driver instrument 24. The driver instrument 24 is initially assembled by inserting the end portion 64 of the drive shaft 26 into the axial opening 74 in the handle 28 to provide the driver instrument 24 with a hexagonally-shaped driving tip 66. As discussed above, the retention mechanism 76 serves to maintain axial engagement between the shaft 26 and the handle 28.

The portion of the spinal column being treated is accessed from an anterior approach, and if an interbody implant 84 is to be used in association with the surgical procedure, an intervertebral opening or space 86 is formed between the upper and lower vertebrae 80, 82. The interbody implant 84 is inserted into the intervertebral opening 86 and the spinal plate 20 is positioned along an anterior aspect of the spinal column so as to extend between the upper and lower vertebrae 80, 82. The spinal plate 20 may then be secured to the upper and lower vertebrae 80, 82 via engagement of the bone anchors 22 into vertebral bone. Specifically, the hexagonally-shaped driving tip 66 of the driver instrument 24 is inserted into the correspondingly-shaped tool receiving recess 112 formed in the head 102 of the bone screw 22. The threaded shank 100 of the bone screw 22 is inserted into a respective one of the bone anchor openings 90 in the spinal plate 20 at the appropriate angular orientation a relative to the spinal plate 20. The self-cutting shank 100 is then driven into vertebral bone until the spherical-shaped surface 110 of the one screw head 102 is engaged tightly against the spherical-shaped recessed portion 92 of the bone anchor opening 90.

Following engagement of the spinal plate 20 to the upper and lower vertebrae 80, 82, the shaft 26 of the driver instrument 24 is removed from the axial opening 74 in the handle 28 and is re-assembled with the handle 28 by inserting the end portion 62 into axial opening 74, thereby providing the driver instrument 24 with a cross-shaped driving tip 68. Once again, the retention mechanism 76 serves to maintain axial engagement between the shaft 26 and the handle 28. The retaining devices 120 are then engaged against the screw heads 102 to prevent the bone screws 22 from loosening and backing out. Specifically, the cross-shaped driving tip 68 of the driver instrument 24 is inserted into the correspondingly-shaped tool receiving recess 126 in the fastener 122, and the fastener 122 is threadingly advanced into the threaded aperture (not shown) in the spinal plate 20 until the washer 124 abuts against the screw head 102. Threading advancement of the fastener 122 through the spinal plate 20 may result in engagement of the fastener 122 with vertebral bone, thereby further securing the spinal plate 20 to the upper and lower vertebrae 80, 82.

While the invention has been illustrated and described in detail in the drawings and foregoing description, the same is to be considered as illustrative and not restrictive in character, and that all changes and modifications that come within the spirit of the invention are desired to be protected.

1. A surgical kit for use in spinal surgery, comprising:
a spinal implant;
instrumentation adapted for use in association with the spinal surgery; and
packaging adapted to contain and maintain said spinal implant and said instrumentation in a sterilized condition prior to the spinal surgery.

2. The surgical kit of claim 1, wherein said spinal implant is adapted for engagement between first and second vertebrae.

3. The surgical kit of claim 2, wherein said spinal implant comprises an elongate member and a number of bone anchors adapted to secure said elongate member to the first and second vertebrae.

4. The surgical kit of claim 3, wherein said elongate member comprises a spinal plate and wherein said bone anchors comprise bone screws.

5. The surgical kit of claim 1, wherein said spinal implant comprises an interbody implant adapted for disposition within an intervertebral space between first and second vertebrae.

6. The surgical kit of claim 5, further comprising a bone growth promoting substance for disposition between the first and second vertebrae to facilitate fusion.

7. The surgical kit of claim 1, wherein said instrumentation is designed for planned disposal.

8. The surgical kit of claim 1, wherein said instrumentation is designed for use in association with a limited number of spinal surgeries.

9. The surgical kit of claim 8, wherein said instrumentation is designed for use in association with a single spinal surgery.

10. The surgical kit of claim 1, wherein at least a portion of said instrumentation is subject to degradation upon exposure to a sterilization procedure.

11. The surgical kit of claim 10, wherein said degradation comprises deformation.

12. The surgical kit of claim 10, wherein said degradation comprises discoloration.

13. The surgical kit of claim 10, wherein said degradation occurs gradually upon exposure to multiple sterilization procedures.

14. The surgical kit of claim 10, wherein said degradation occurs immediately upon exposure to a single sterilization procedure.

15. The surgical kit of claim 10, wherein said sterilization procedure comprises autoclaving.

16. The surgical kit of claim 10, wherein said degradation causes said instrumentation to become substantially inoperative.

17. The surgical kit of claim 1, wherein said instrumentation comprises a first portion and a second portion, said first portion being selectively engagable with said second portion.

18. The surgical kit of claim 17, wherein said first portion of said instrumentation comprises a shaft, said second portion of said instrumentation comprising a handle, said shaft including opposite first and second end portions, said first and second end portions being reversible relative to said handle, said first end portion adapted to perform a first function associated with the spinal surgery, said second end portion adapted to perform a second function associated with the spinal surgery.

19. The surgical kit of claim 18, wherein said first end portion comprises a first tip configuration adapted for engagement with a first element associated with said spinal implant, said second end portion comprising a second tip configuration adapted to for engagement with a second element associated with said spinal implant.

20. The surgical kit of claim 1, wherein said instrumentation is configured to perform multiple functions associated with the spinal surgery.

21. The surgical kit of claim 1, wherein said packaging is adapted to integrally contain said spinal implant and said instrumentation.

22. The surgical kit of claim 21, wherein said packaging includes a plurality of compartments sized to receive respective components of said spinal implant and said instrumentation therein.

23. The surgical kit of claim 1, wherein said packaging is formed of a material capable of providing direct visualization of said spinal implant and said instrumentation contained therein.

24. The surgical kit of claim 1, wherein said packaging comprises an inner container and outer container, said inner container adapted to contain and maintain said spinal implant and said instrumentation in said sterilized condition, said outer container adapted to contain and maintain said inner container in a sterilized condition prior to the spinal surgery.

25. The surgical kit of claim 24, wherein said outer container includes a first removable seal to provide selective access to said inner container, said inner container including a second removable seal to provide selective access to said spinal implant and said instrumentation contained therein.

26. The surgical kit of claim 1, further comprising a template including a number of images corresponding to one or more select sizes of said spinal implant, one of said template images corresponding to a size of said spinal implant included with the surgical kit.

27. The surgical kit of claim 26, wherein said template is provided external to said packaging to provide access to said template without compromising said sterilized condition of said spinal implant and said instrumentation.

28. The surgical kit of claim 26, wherein said template includes an indication of a magnification factor associated with said template images.

29. The surgical kit of claim 1, wherein the surgical kit is self-contained to include all surgical equipment required to perform a designated spinal surgery.

30. A surgical kit for use in spinal surgery, comprising:
a surgical equipment set, including:
a spinal plate;
a number of bone screws adapted to secure said spinal plate to first and second vertebrae; and
a driver instrument adapted to drive said bone screws into engagement with vertebral bone; and
packaging adapted to contain and maintain said surgical equipment set in a sterilized condition prior to the spinal surgery.

31. The surgical kit of claim 30, wherein said bone screws are self-cutting bone screws.

32. The surgical kit of claim 30, wherein said bone screws are variable-angle screws.

33. The surgical kit of claim 30, wherein said surgical equipment set includes an interbody implant adapted for disposition within an intervertebral space between the first and second vertebrae.

34. The surgical kit of claim 30, wherein said driver instrument is designed for planned disposal.

35. The surgical kit of claim 30, wherein at least a portion of the driver instrument is subject to degradation upon exposure to a sterilization procedure.

36. The surgical kit of claim 30, wherein said driver instrument extends generally along a longitudinal axis and comprises a shaft portion and a handle portion, said handle portion including a first portion rotatably coupled to a second portion to provide relative rotational movement therebetween about the longitudinal axis.

37. The surgical kit of claim 30, wherein said driver instrument comprises a shaft portion and a handle portion, said shaft portion being selectively engagable with said handle portion.

38. The surgical kit of claim 37, wherein said shaft portion includes opposite first and second end portions, said first and second end portions being reversible relative to said handle portion, said first end portion comprises a first tip configuration adapted for engagement with said bone screws, said second end portion comprising a second tip configuration adapted to for engagement with a second element associated with said spinal plate.

39. The surgical kit of claim 30, further comprising a template including a number of images corresponding to one or more select sizes of said spinal plate, one of said template images corresponding to a size of said spinal plate included with the surgical kit, said template being provided external to said packaging to provide access to said template without compromising said sterilized condition of said spinal plate and said instrumentation.

40. A method of providing sterilized surgical equipment for use in spinal surgery, comprising:
providing a surgical equipment set including a spinal implant and instrumentation adapted for use in association with the spinal surgery;
packaging the surgical equipment set within a sealed container;
sterilizing the surgical equipment set; and
delivering the surgical equipment set to a site for performing the spinal surgery.

41. The method of claim 40, wherein the spinal surgery comprises engaging the spinal implant between first and second vertebrae.

42. The method of claim 41, wherein the spinal implant comprises an elongate member, the engaging comprising securing the elongate member to the first and second vertebrae with a plurality of bone anchors.

43. The method of claim 41, wherein the spinal implant comprises an interbody implant, the engaging comprising inserting the interbody implant within an intervertebral space between the first and second vertebrae.

44. The method of claim 41, wherein the surgical equipment set includes a bone growth promoting substance for disposition between the first and second vertebrae to facilitate fusion.

45. The method of claim 40, further comprising providing a template including a number of images corresponding to one or more select sizes of the spinal implant, one of the template images corresponding to a size of the spinal implant included with the surgical equipment set.

46. The method of claim 45, further comprising:
comparing the template images to a visual representation of a portion of the spinal column subjected to the spinal surgery; and
selecting a surgical equipment set including a spinal implant having a size suitable for use in association with the spinal surgery.

47. The method of claim 46, further comprising providing the template with an indication corresponding to a magnification factor associated with the template images; and
selecting a magnification factor associated with the visual representation corresponding to the magnification factor associated with the template images.

48. The method of claim 45, wherein the template is provided external to the sealed container, the method further comprising accessing the template without compromising the sterility of the surgical equipment set.

49. The method of claim 40, further comprises disposing of the instrumentation after a select number of spinal surgeries.

50. The method of claim 49, wherein the disposing occurs upon completion of a single spinal surgery.

51. The method of claim 40, wherein the instrumentation comprises separate first and second portions, the method further comprising assembling the first portion with the second portion subsequent to the delivering.

52. The method of claim 51, wherein the first portion of the instrumentation comprises a handle, the second portion of the instrumentation comprising a shaft including opposite first and second end portions, the method further comprising:
performing a first function with the first end portion of the shaft;
reversing the positions of the first and second end portions of the shaft relative to the handle; and
performing a second function with the second end portion of the shaft.

53. The method of claim 40, wherein the sealed container includes an inner sealed container and an outer sealed container; and
wherein the packaging comprises packaging the surgical equipment set within the inner sealed container and packaging the inner sealed container within the outer sealed container.

54. The method of claim 40, wherein the providing includes selecting a spinal implant having a particular size from a select range of sizes, the select range of sizes being based on a predicted spinal implant usage criteria.

55. The method of claim 40, further comprising providing the surgical equipment set with all surgical components required to perform a designated spinal surgery.
